(19) United States
(12) Patent Application Publication
(10) Pub. No.: US 2016/0106398 A1
(43) Pub. Date: Apr. 21, 2016

(54) System and Method to Identify Abnormal Menstrual Cycles and Alert the User

(71) Applicant: Narmadha Kuppuswami, Wheaton, IL (US)

(72) Inventor: Narmadha Kuppuswami, Wheaton, IL (US)

(21) Appl. No.: 14/880,588

(22) Filed: Oct. 12, 2015

Related U.S. Application Data
(60) Provisional application No. 62/064,035, filed on Oct. 15, 2014.

(51) Int. Cl. A61B 10/00; A61B 5/00; G06F 19/00; A61B 5/11

(52) U.S. Cl. CPC A61B 10/0012; A61B 5/1112; A61B 5/486; A61B 5/7282; A61B 5/746; A61B 5/747; G06F 19/345; A61B 2010/0029; A61B 2560/0475

(57) ABSTRACT

A system and method to identify abnormal menstrual cycles and alert the user comprising an electronic device running a software application. The software application includes a method that processes and analyzes data and determines when the user exhibits signs and symptoms that require medical attention. The method receives, compiles, and analyzes information regarding at least one of the user's general symptoms, number of days bleeding, mood changes, heavy menstrual flow, inter-menstrual bleeding, and bleeding after sex. The method determines and alerts the user when the information, signs and/or symptoms the user is experiencing indicate that the user's menstrual cycle may be critically abnormal or that the user may have a heavy abnormal period and alerts the user regarding the severity, prompting the user to call her doctor as soon as possible. The method further determines and alerts the user when the user should seek medical attention and contact her doctor at the phone number the user provided upon downloading the software application. If the user did not specify her doctor's phone number or address, the system will use GPS technology to provide a list of all gynecologists and primary care physicians in close proximity to the user's location. The method will also provide the user with a call button to directly call the doctor from within the software application. Additionally, if the user consents, the system and method can capture and compile patient information and statistics and build a database to be used for research purposes.

### CROSS REFERENCE

This application claims priority to provisional patent application Ser. No. 62/064,035, filed Oct. 15, 2014, to the extent allowed by law.

### TECHNICAL FIELD

The present disclosure relates to a system and method designed to track menstrual cycles, and more particularly, to a mobile application to track symptoms, identify abnormal menstrual cycles, and alert the user when an abnormal menstrual cycle has been detected.

### BACKGROUND

Women typically monitor their menstrual cycles from day one of their last menstruation to determine future menstruation, fertility, ovulation, and physical and mental symptoms. At times, the demands of daily life, family, and work make it difficult to keep track of various dates and overlook adverse symptoms that may indicate signs of abnormal menstrual cycles and health risk. Several websites and mobile applications have emerged to aide in a woman's desire to monitor their menstrual cycles, to identify fertile periods, and to plan social events around their menstrual cycles, thus providing a method for collecting and storing patient data. However, these websites and mobile applications do not provide a method for the analysis patient data and symptoms, detection of adverse symptoms, and identify abnormal menstrual cycles to provide a recommendation that the patient should seek medical attention.

U.S. Pat. No. 8,632,474 discloses a system and method for providing information based on menstrual data that includes collecting personal data including menstrual data regarding a menstrual cycle on a woman from a user, identifying a phase within the menstrual cycle, storing predetermined information, selecting information in storage, which is personalized based on the phase identified, and delivering the personalized information. Patent '474 does not disclose a method that analyzes menstrual data to detect adverse symptoms of abnormal menstrual cycles to provide a recommendation that the user should seek medical attention.

U.S. patent publication 2010/0125186 discloses a medical diagnostic and communication system that includes, in some embodiments, an input device operable for a patient to enter primary symptoms being experienced, a processing device operable for processing the primary symptoms to form a provisional diagnosis, and an output device operable for providing the provisional diagnosis to the patient. In other embodiments, the system includes an input device operable for a patient to enter medical history and primary symptoms being experienced, a processing device operable for interactively querying the patient based on the medical history and the primary symptoms to determine if additional symptoms are being experienced, a communication device operable for communicating with a physician's office and an output device operable for providing medical advice to the patient. The medical advice is given by the physician's office when the physician's office is open and by the system when the physician's office is closed. The system in publication '186 serves as a remote waiting room for the patient by providing medical information to the patient's physician without the patient actually having to visit the physician's office and as a surrogate physician when the patient's physician is otherwise unavailable. In the '186 system, the patient accesses the system when they already know that they need medical attention. The '186 system does not serve to alert the patient that they may need to seek medical attention when the patient does not suspect any health issues.

U.S. patent publication 2014/0100885 discloses a method and system for automated medical records processing with cloud computing that includes plural electronic medical templates specifically designed such that they reduce the complexity and risk associated with collecting patient encounter information, creating a medical diagnosis and help generate the appropriate number and type of medical codes for a specific type of medical practice when processed. The medical codes and other types of processed patient encounter information are displayed in real-time on electronic medical records and invoices immediately after a patient encounter via a cloud computing network. Publication '885 does not disclose a system that is to be used by the user to analyze menstrual data to detect adverse symptoms of abnormal menstrual cycles to provide a recommendation that the user should seek medical attention.

For the foregoing reasons, there is a need for a system and method for the analysis patient data and symptoms, detection of adverse symptoms, and identify abnormal menstrual cycles to provide a recommendation that the patient should seek medical attention.

### SUMMARY

The system and method to identify abnormal menstrual cycles and alert the user of an illustrated embodiment of the present disclosure comprises an electronic device running a software application. The software application includes a method that processes and analyzes data and determines whether the user exhibits signs and symptoms that require medical attention. The method receives, compiles, and analyzes information regarding at least one of the user's general symptoms, number of days bleeding, mood changes, heavy menstrual flow, inter-menstrual bleeding, and bleeding after sex. The method determines and alerts the user when the information, signs and/or symptoms the user is experiencing indicate that the user's menstrual cycle may be critically abnormal or that the user may have a heavy abnormal period that may require immediate medical attention. Based on the signs and symptoms that the user provides, the method further determines and alerts the user that the user's menstrual cycle may be critically abnormal and should seek medical attention as soon as possible. Additionally, the system and method can capture and compile patient information and statistics, with the user's approval, to build a database for research purposes.

### DESCRIPTION OF THE DRAWINGS

These and other features, aspects, and advantages of the present disclosure will become better understood with regard to the following description, appended claims, and accompanying drawings where:

FIG. 1 is a flow diagram analyzing symptoms, number of days of bleeding, and mood changes to determine whether the user should seek medical attention in an illustrated embodiment of the system and method of the present disclosure; and

FIG. 2 is a flow diagram analyzing heavy flow days, inter-menstrual bleeding, and bleeding after sex to determine whether the user should seek medical attention in the illustrated embodiment of the system and method of the present disclosure.

### DETAILED DESCRIPTION

The system and method to identify abnormal menstrual cycles and alert the user of an illustrated embodiment of the present disclosure comprises an electronic device running a software application. The user downloads and installs a software application onto her electronic device and enters information relating to her menstrual cycles as she desires. The software application includes a method that processes and analyzes data and determines whether the user exhibits signs and/or symptoms that require medical attention.

A primary object of the present disclosure is to provide a system and method that monitors a woman's menstrual cycles.

It is another object of the present disclosure to provide a system and method that collects and stores a woman's medical data, medical history, and menstrual cycle information.

It is yet another object of the present disclosure to provide a system and method that analyzes menstrual cycle data, patient data, and patient symptoms.

It is yet another object of the present disclosure to provide a system and method that detects abnormal symptoms and/or abnormal combinations of symptoms

It is yet another object of the present disclosure to provide a system and method that alerts the user that, based on her symptoms, menstrual cycle data, and personal data, she should seek medical attention.

It is yet another object of the present disclosure to provide a system and method that captures and compiles patient information and statistics, with the patient's approval, building a database for research purposes.

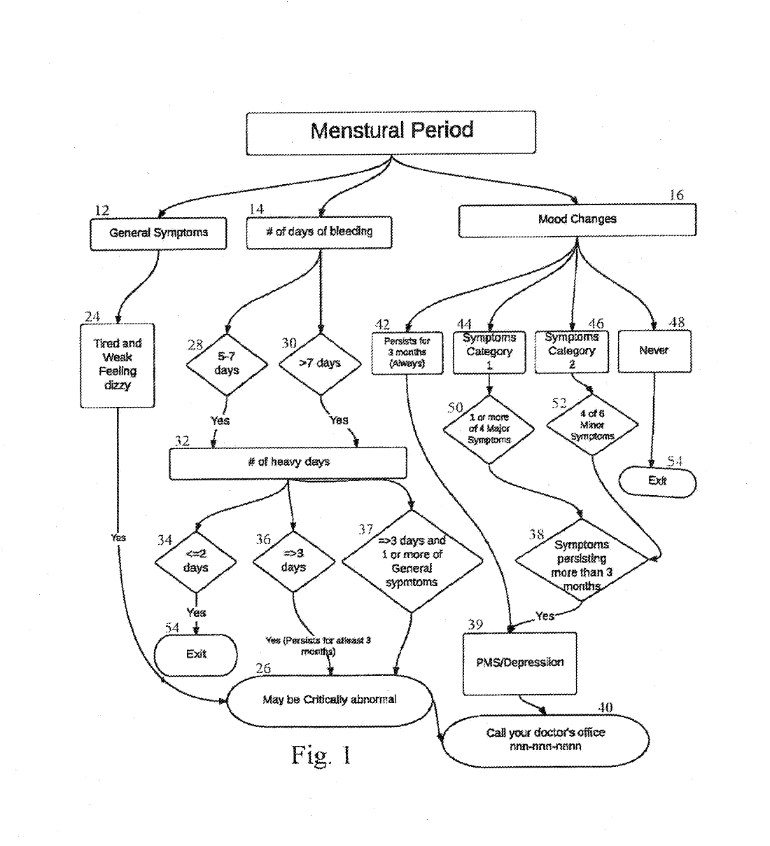

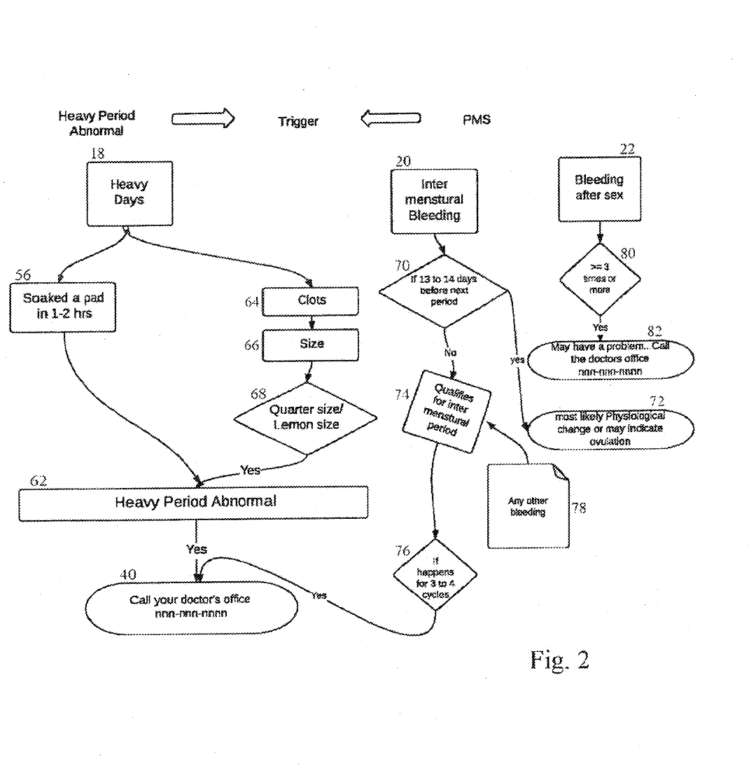

The flow diagram of the method of the illustrated embodiment of the present disclosure is shown completely by combining FIG. 1 and FIG. 2, FIG. 1 positioned to the left of FIG. 2. The method, shown in FIGS. 1 and 2, begins by allowing the user to enter data relating to the following categories: general symptoms 12; number of days bleeding 14; mood changes 16; heavy days 18; inter-menstrual bleeding 20; and bleeding after sex 22. Each category allows the method to determine whether the user's signs and/or symptoms indicate that her menstrual cycle may be abnormal. Referring to FIG. 1, under general symptoms 12, if the user indicates that she is feeling tired and weak and/or feeling dizzy 24, the method will alert the user that her menstrual cycle may be critically abnormal 26. General symptoms 12 can include any of the major symptoms (sudden onset of being sad, tearful irritable, or angry; persistent marked anger or irritability; marked anxiety or tension; and feelings of hopelessness) or any of the minor symptoms (decreased interest in usual activity; marked lack of energy or fatigue; unable to concentrate; marked change in appetite, overeating or food cravings; difficulty in sleeping or over-sleepiness; and breast tenderness, headaches, achiness all over, and weight gain). Under this first category, if the user indicates that she is feeling tired, weak, and/or dizzy 24, the method will alert the user that her menstrual cycle may be critically abnormal 26 and will instruct the user to call her doctor's office 40 at the phone number the user provided upon installing the software application.

Under the second category indicating the number of days bleeding 14, the user can select five to seven days 28 or greater than seven days 30. Under either five to seven days 28 or greater than seven days 30, the user is prompted to enter the number of heavy days 32 she had. If the user indicates that her number of heavy days were less than or equal to two days 34, the method will exit 54, as no abnormal conditions exist. If the user indicates that her number of heavy days were equal to or greater than three days 36, the method will alert the user that her menstrual cycle may be critically abnormal 26 and her symptoms have persisted more than three months, the method will alert the user that her menstrual cycle may be critically abnormal 26 and will instruct the user to call her doctor's office 40 at the phone number the user provided upon installing the software application. If the user indicates that her number of heavy days were equal to or greater than three days and the user is experiencing one or more general symptoms 37, the method will alert the user that her menstrual cycle may be critically abnormal 26 and will instruct the user to call her doctor's office 40 at the phone number the user provided upon installing the software application. If the user did not specify her doctor's phone number or address, the system will use global positioning system (GPS) technology to provide a list of all gynecologists and primary care physicians in close proximity to the user's location. The method will also provide the user with a call button to directly call the doctor from within the software application.

Under the third category indicating mood changes 16, the user can select from always 42 (symptoms persisting throughout the entire three month period), symptoms category 1 (major symptoms) 44, symptoms category 2 (minor symptoms) 46, and never 48 in which case the method will exit. Symptoms category 1 includes major symptoms: sudden onset of being sad, tearful irritable, or angry; persistent marked anger or irritability; marked anxiety or tension; and feelings of hopelessness. Symptoms category 2 includes minor symptoms: decreased interest in usual activity; marked lack of energy or fatigue; unable to concentrate; marked change in appetite, overeating or food cravings; difficulty in sleeping or over-sleepiness; and breast tenderness, headaches, achiness all over, and weight gain. If the user indicates that her mood always changes 42, meaning that she mood changes have persisted for at least three months, the method will alert the user that her menstrual cycle may be complicated by PMS/depression 39 and will alert the user to call her doctor's office 40 at the phone number the user provided upon installing the software application or provide the phone number of a doctor using GPS technology. If the user indicates that she exhibits one or more of the four major symptoms 50 from symptoms category 1 44 and the symptoms have persisted more than three months 38, the method will alert the user that her menstrual cycle may be complicated by PMS/depression 39 and will alert the user to call her doctor's office 40 at the phone number the user provided upon installing the software application or provide the phone number of a doctor using GPS technology. If the user indicates that she has not experienced any of the major symptoms, the method will exit. If the user indicates that she exhibits at least four of the six minor symptoms 52 from symptoms category 2 46 and the symptoms have persisted more than three months 38, the method will alert the user that her menstrual cycle may be complicated by PMS/depression 39 and will alert the user to call her doctor's office 40 at the phone number the user provided upon installing the software application or provide the phone number of a doctor using GPS technology. If the user indicates that she never experiences mood changes 48, the method will exit 54 as no abnormal conditions exist. If the user has not experienced at least four minor symptoms, the method will exit.

Referring to FIG. 2, under the fourth category indicating heavy days 18, if the user indicates that her feminine product was soaked in one to two hours 56, the method will alert the user that she has a heavy, abnormal period 62 and will alert the user to call her doctor's office 40 at the phone number the user provided upon installing the software application. If the user indicates that she has experienced clots 64 the size 66 from a quarter to a lemon 68, the method will alert the user that she may have a heavy, abnormal period 62 and will alert the user to call her doctor's office 40 at the phone number the user provided upon installing the software application or provide the phone number of a doctor using GPS technology.

Under the fifth category indicating inter-menstrual bleeding 20, if the user indicates that she has experienced inter-menstrual bleeding 13 to 14 days before her next period 70, the method will indicate that the bleeding may be physiological and will alert the user that she is most likely experiencing a physiological change or that the bleeding may indicate ovulation 72. If the user is experiencing inter-menstrual bleeding that is not 13 to 14 days before her next period 70 or any is experiencing any other bleeding 78, then the bleeding qualifies as an inter-menstrual period 74 and if she experiences inter-menstrual periods for three to four cycles 76, the method will alert the user to call her doctor's office 40 at the phone number the user provided upon installing the software application. If she experiences inter-menstrual bleeding for less than three cycles, the method will exit.

Under the sixth category indicating bleeding after sex 22, if the user indicates that she has experienced bleeding after sex three or more times 80, the method will alert the user that she may have a problem 82 and will alert the user to call her doctor's office 40 at the phone number the user provided upon installing the software application or provide the phone number of a doctor using GPS technology. If the user indicates that she has not experienced bleeding after sex or has experienced bleeding after sex less than three times, the method will exit.

The system and method of the present disclosure is primarily used to alert the user that, based on their symptoms, menstrual cycle data, and patient data, they should seek medical attention. Additionally, if the user consents, the system and method can capture and compile patient information and statistics and build a database to be used for research purposes. The user can download her patient information and statistics in a format to be forwarded to her doctor's office either via email or via paper document.

The foregoing description of an illustrated embodiment of the disclosure has been presented for purposes of illustration and description, and is not intended to be exhaustive or to limit the disclosure to the precise form disclosed. The description was selected to best explain the principles of the disclosure and practical application of these principles to enable others skilled in the art to best utilize the disclosure in various embodiments and various modifications as are suited to the particular use contemplated. It is intended that the scope of the disclosure not be limited by the specification, but be defined by the claims set forth below.

What is claimed is:

1. A method for the diagnosis of abnormal menstrual cycles of a user accomplished via a software application on an electronic device computer, comprising the steps of:
a. receiving data based on at least one of the user's general symptoms, number of days the user is bleeding, mood changes, heavy menstrual flow, inter-menstrual bleeding, and bleeding after sex the user is experiencing;
b. compiling and analyzing the data;
c. determining and alerting the user when at least one of the user's menstrual cycle may be critically abnormal, the user's menstrual cycle symptoms may be critically abnormal, and the user may have a heavy abnormal period; and
d. determining and alerting the user when the user should call a doctor and seek medical attention.

2. The method of claim 1, wherein the general symptoms comprise at least one of:
a. a sudden onset of being at least one of sad, tearful, irritable, and angry;
b. one of persistent marked anger and persistent marked irritability;
c. one of marked anxiety and marked tension;
d. feelings of hopelessness;
e. a decreased interest in usual activity;
f. at least one of marked lack of energy and marked fatigue;
g. an inability to concentrate;
h. at least one of a marked change in appetite, overeating, and food cravings;
i. one of difficulty sleeping and over-sleepiness; and
j. at least one of breast tenderness, headaches, all-over achiness, and weight gain.

3. The method of claim 1, further comprising the steps of:
a. identifying when the data indicates that the user's general symptoms include feeling at least one of tired, weak, and dizzy;
b. determining and alerting the user that the user's menstrual cycle may be critically abnormal; and
c. notifying the user to call her doctor's office.

4. The method of claim 1, further comprising the steps of:
a. identifying when the data indicates that the user has experienced at least one of a range of five to seven days of bleeding and more than seven days of bleeding;
b. identifying when the data indicates that the user has experienced at least three days of heavy bleeding for at least three months;
c. determining and alerting the user that the user's menstrual cycle may be critically abnormal; and
d. notifying the user to call her doctor's office.

5. The method of claim 2, further comprising the steps of:
a. identifying when the data indicates that the user has experienced at least one of a range of five to seven days of bleeding and more than seven days of bleeding;
b. identifying when the data indicates that the user has experienced at least three days of heavy bleeding and has experienced at least one general symptom;
c. determining and alerting the user that the user's menstrual cycle may be critically abnormal; and
d. notifying the user to call her doctor's office.

6. The method of claim 1, further comprising the steps of:
a. identifying when the data indicates that the user has experienced mood changes for at least three months;
b. determining and alerting the user that she is experiencing at least one of pre-menstrual syndrome (PMS) and depression; and
c. notifying the user to call her doctor's office.

7. The method of claim 1, further comprising the steps of:
a. identifying when the data indicates that the user has experienced at least one of a sudden onset of being at least one of sad, tearful, irritable, and angry, persistent marked anger, persistent marked irritability, marked anxiety, marked tension, and feelings of hopelessness for at least three months;
b. determining and alerting the user that she is experiencing at least one of PMS and depression; and
c. notifying the user to call her doctor's office.

8. The method of claim 1, further comprising the steps of:
a. identifying when the data indicates that the user has experienced at least four of decreased interest in usual activity, marked lack of energy, marked fatigue, inability to concentrate, marked change in appetite, over-eating, food cravings, difficulty sleeping, over-sleepiness, breast tenderness, headaches, all-over achiness, and weight gain for at least three months;
b. determining and alerting the user that she is experiencing at least one of PMS and depression; and
c. notifying the user to call her doctor's office.

9. The method of claim 1, further comprising the steps of:
a. identifying when the data indicates that the user has experienced heavy days and soaked a feminine product in less than two hours;
b. determining and alerting the user that she is experiencing an abnormal heavy period; and
c. notifying the user to call her doctor's office.

10. The method of claim 1, further comprising the steps of:
a. identifying when the data indicates that the user has experienced heavy days and has had clots at least 15/16 of an inch;
b. determining and alerting the user that she is experiencing an abnormal heavy period; and
c. notifying the user to call her doctor's office.

11. The method of claim 1, further comprising the steps of:
a. identifying when the data indicates that the user has experienced inter-menstrual bleeding for one of thirteen and fourteen days before her next menstrual cycle; and
b. notifying the user at least one of she is most likely experiencing a physiological change and the inter-menstrual bleeding may indicate ovulation.

12. The method of claim 1, further comprising the steps of:
a. identifying when the data indicates at least one of the user has experienced inter-menstrual bleeding that is not one of thirteen and fourteen days before her next menstrual cycle and the user has experienced any bleeding;
b. determining and alerting the user that she is experiencing inter-menstrual bleeding; and
c. notifying the user to call her doctor's office when the inter-menstrual bleeding occurs for at least three menstrual cycles.

13. The method of claim 1, further comprising the steps of:
a. identifying when the data indicates that the user has experienced bleeding after sex more at least three times;
b. determining and alerting the user that she may have a problem; and
c. notifying the user to call her doctor's office.

14. The method of claim 1, further comprising the steps of:
a. receiving a phone number for the doctor; and
b. storing the phone number.

15. The method of claim 14, further comprising the steps of:
a. displaying the phone number on the electronic device computer; and
b. providing a call button display adapted to allow the user to directly call the doctor from within the software application.

16. The method of claim 1, further comprising the steps of:
a. determining the user's current location based on a GPS signal received by the electronic device computer;
b. identifying one of a gynecologist office and a primary care physician office nearest to the user's current location;
c. determining a phone number of one of the gynecologist office and primary care physician office;
d. displaying the phone number on the electronic device computer; and
e. providing a call button display adapted to allow the user to directly call the doctor from within the software application.

17. The method of claim 1, further comprising the step of:
a. compiling and saving at least one of patient information, patient statistics, and the data in a database;
b. downloading and saving at least one of the patient information, the patient statistics, and the data from the database into a file; and
c. sending the file to the user's doctor's office via at least one of email and physical mail.

18. A system for the diagnosis of abnormal menstrual cycles of a user accomplished via a software application on an electronic device computer, the system comprising:
a. a memory;
b. an interface adapted to be used on the electronic device computer to send and receive data between the electronic device computer and the user; and
c. a GPS receiver installed in the electronic device computer, the GPS receiver adapted to receive a GPS signal;
d. a processor configured to execute instructions in the software application to:
e. receive data based on at least one of the user's general symptoms, number of days the user is bleeding, mood changes, heavy menstrual flow, inter-menstrual bleeding, and bleeding after sex the user is experiencing;
f. compile and analyze the data;
g. determine and alert the user when at least one of the user's menstrual cycle may be critically abnormal, the user's menstrual cycle symptoms may be critically abnormal, and the user may have a heavy abnormal period; and
h. determine and alert the user when the user should call a doctor and seek medical attention.

19. The system of claim 18, wherein the processor is configured to execute instructions in the software application to:
a. determine the user's current location based on the GPS signal;
b. identify one of a gynecologist office and a primary care physician office nearest to the user's current location;
c. determine a phone number of one of the gynecologist office and the primary care physician office;
d. display the phone number on the interface; and
e. provide a call button display on the interface, the call button display adapted to allow the user to directly call the doctor from within the software application.

20. The system of claim 18, wherein the processor is configured to execute instructions in the software application to:
a. compile and store at least one of patient information, patient statistics, and the data in a database;
b. download and save at least one of the patient information, the patient statistics, and the data from the database into a file; and
c. send the file to the user's doctor's office via at least one of email and physical mail.
